(19) United States
(12) Patent Application Publication
(10) Pub. No.: US 2014/0296147 A1
(43) Pub. Date: Oct. 2, 2014

(54) METHODS OF TREATING OR PREVENTING BLOOD LOSS DURING SURGERY USING THE SERINE PROTEASE INHIBITOR MDCO-2010

(71) Applicant: THE MEDICINES COMPANY, Parsippany, NJ (US)

(72) Inventors: Andreas Van De Locht, Munich (DE); Wulf Dietrich, Munich (DE); Lars Englberger, Heimberg (CH); Peter Villiger, Hoboken, NJ (US); John Villiger, St Heliers (NZ)

(21) Appl. No.: 14/349,803

(22) PCT Filed: Oct. 5, 2012
(86) PCT No.: PCT/US12/58859

§ 371 Date: Apr. 4, 2014

Related U.S. Application Data
(60) Provisional application No. 61/544,215, filed on Oct. 6, 2011.
(60) Provisional application No. 61/547,499, filed on Oct. 14, 2011.

(51) Int. Cl. A61K 38/05

(52) U.S. Cl. CPC A61K 38/05
USPC 514/14.4; 514/13.7

(57) ABSTRACT

Methods of inhibiting, treating or preventing blood loss in a subject undergoing surgery through the administration of an effective amount of the novel serine protease inhibitor MDCO-2010 or its salts or esters are disclosed.

### BACKGROUND OF THE INVENTION

Many patients experiencing cardiac and thoracic surgery require cardiopulmonary bypass (CPB). CPB is used to maintain blood flow and to facilitate tissue oxygenation during surgical procedures with cardioplegia, such as coronary artery bypass grafting, cardiac valve replacement or repair, thoracic aortic aneurysm repair, and heart or heart/lung transplantation. CPB and its concomitant therapies of hemodilution, hypothermia, anticoagulation, and cardioplegia can result in dramatic fluid and electrolyte imbalances, catecholamine storm, and hemorrhage. In particular, perioperative bleeding is a serious complication that adversely affects the morbidity and mortality of cardiac surgery (Koch et al. (2006) Ann Thorac Surg 81: 1650-7).

Blood contact with nonendothelial surfaces of the cardiopulmonary bypass initiates clotting by generation of thrombin. Thus, an anticoagulant is required for extracorporeal circulation. Standard, unfractionated heparin (UFH) is almost exclusively used, but it is not entirely satisfactory (Edmunds & Colman (2006) Ann Thorac Surg 82: 2315-22). CPB and other applications of extracorporeal circulation—e.g. ventricular assist devices, extracorporeal membrane oxygenation, dialysis—result in activation of platelets and monocytes. Both conversions lead to a slow but steady accumulation of Factor Xa that subsequently converts prothrombin to thrombin. Thus, heparin permits the continuous generation of thrombin which induces a myriad of pro-inflammatory effects, likely contributing to the occurrence of systemic inflammatory response syndrome (SIRS) in patients subjected to CPB.

Thrombin generation also leads to plasmin formation and fibrinolysis. Such simultaneous generation of thrombin and plasmin results in a consumptive coagulopathy (Marder et al. (2006). Consumptive thrombohemorrhagic disorders. In: Hemostasis and Thrombosis, 5th ed. Colman et al eds. Philadelphia, Pa.: Lippincott, Williams & Wilkins: 1571-600). Consequently, both F1+2 and D-dimer progressively increase during CPB and peak with the administration of protamine (Chandler & Velan (2004) Blood Coag Fibrinol 15: 583-91).

Such impaired haemostasis is a major sequela of cardiac surgery with CPB, because it frequently leads to excessive blood loss, requires blood product transfusions and is linked to adverse outcomes (Karkouti et al. (2004) Transfusion 44: 1453-62).

Furthermore, current cardiological therapeutic strategies require extensive treatment with potent platelet inhibitors such as tirofiban or clopidogrel (Jennings L K (2005) Expert Opin Drug Metab Toxicol 1: 727-37). These treatments increase the demand for blood and blood products as well as for coagulation factors. Despite all technical and tactical advances, cardiac surgical procedures continue to show an increasing tendency to consume allogeneic blood products currently being estimated to account for around 20% of a western industrial nation's supply (DeAnda et al. (2006) Am J Med Qual 21: 230-7).

To alleviate this complication, prophylactic antifibrinolytic therapies are now widely accepted as a strategy to inhibit excessive fibrinolysis. The most thoroughly evaluated antifibrinolytic agent is aprotinin, a bovine-derived broad spectrum serine protease inhibitor. Aprotinin was shown to consistently reduce blood loss and transfusion requirements, and it was the only antifibrinolytic agent associated with reduced mortality, a decreased incidence of stroke, and shortened hospital stay. Many surgeons became convinced of its superiority in comparison with the synthetic lysine analogs. However, as its use spread, concerns regarding renal toxicity and safety issues surfaced (Henry et al. (2009) CMAJ 180: 183-93). In November 2007, a randomized, controlled trial, BART, designed to settle efficacy issues, was stopped by the trial Data Safety Monitoring Board. Subsequently, the marketing of aprotinin was stopped.

The two synthetic lysine analogs E-aminocaproic acid and tranexamic acid (TA) are being used alternatively to control bleeding in cardiac surgery. Both have an identical mechanism of action by blocking the activation of plasmin. The toxicity of both drugs is considered low; however, a recent single, retrospective study found a higher incidence of seizures and atrial fibrillation associated with high doses of TA (>4 g) (Murkin et al. (2010) Anesth Analg 110: 350-3). Previous experience has not reported these complications, but concerns have been raised regarding possible renal toxicity in patients who undergo CPB, which is also associated with renal injury independent of either drug.

Therefore, there is a significant unmet need for an effective therapeutic option with an improved safety profile.

### SUMMARY OF THE INVENTION

MDCO-2010 is a synthetic, small molecule (molecular mass of 698 Dalton) that is being developed to reduce blood loss associated with CPB during CABG surgery (Dietrich et al. (2009) Anesthesiology 110:123-30; U.S. Pat. No. 8,207,378). It is a direct, active site inhibitor of plasmin and plasma kallikrein. Thus, it binds to the active site of these enzymes and blocks their proteolytic activity with no requirement for a cofactor. In that respect, its structural mode of action is similar to aprotinin. Both primary targets of MDCO-2010, plasmin and plasma kallikrein are involved in impaired hemostasis.

In vitro and in vivo, MDCO-2010 has demonstrated antifibrinolytic potency and efficacy which is similar to or better than that of aprotinin, along with moderate anticoagulant activity. Compared to aprotinin, MDCO-2010 inhibits additional proteases involved in coagulation activation, Factor Xa and Factor XIa, resulting in moderate anticoagulant properties which do not appear to compromise antifibrinolytic efficacy but which may reduce the risk of thrombotic events.

From a safety perspective, the risks of allergic reactions and prion transmission associated with aprotinin do not arise with MDCO-2010 since it is a synthetic small molecule. Furthermore, MDCO-2010, unlike the protein aprotinin, is not expected to accumulate in the kidney and hence renal toxicity is considered to be unlikely. Finally, MDCO-2010 has a rapid onset and offset of pharmacological action and a short plasma half-life, properties which are suited to its short-term use during a surgical procedure.

The profile of MDCO-2010 to date therefore indicates a potential for significant advantages compared with existing therapy.

In a first embodiment, the present invention is directed to methods of inhibiting, treating or preventing blood loss in a subject undergoing surgery, comprising administering to a subject in need thereof a therapeutically effective amount of MDCO-2010, or a pharmaceutically acceptable salt or ester thereof, thereby inhibiting, treating or preventing blood loss in the subject undergoing surgery.

In certain aspects of this embodiment, the therapeutically effective amount of MDCO-2010, or the salt or ester thereof, administered to the subject is at least about 1 μg/kg/h, at least about 10 μg/kg/h, at least about 30 μg/kg/h, about 12.5 μg/kg/h, about 25 μg/kg/h, about 62.5 μg/kg/h, between about 1 and 500 μg/kg/h, between about 10 and 250 μg/kg/h, between about 10 and 200 μg/kg/h or between about 10 and 100 μg/kg/h of MDCO-2010, or the salt or ester thereof.

In certain aspects of this embodiment, MDCO-2010, or the salt or ester thereof, is administered to the subject via infusion, such as continuous infusion.

In an alternative aspect of this embodiment, the therapeutically effective amount administered to the subject is a bolus dose of MDCO-2010, or the salt or ester thereof, followed by a maintenance dose of MDCO-2010, or the salt or ester thereof. In this alternative aspect, the bolus dose is independently at least about 1 μg/kg, at least about 5 μg/kg, at least about 10 μg/kg, about 5 μg/kg, about 11 μg/kg, about 27 μg/kg, between about 5 and 100 μg/kg, or between about 5 and 50 μg/kg of MDCO-2010, or the salt or ester thereof. The maintenance dose is independently at least about 1 μg/kg/h, at least about 10 μg/kg/h, at least about 30 μg/kg/h, about 12.5 μg/kg/h, about 25 μg/kg/h, about 62.5 μg/kg/h, between about 10 and 200 μg/kg/h, or between about 10 and 100 μg/kg/h of MDCO-2010, or the salt or ester thereof.

In a second embodiment, the present invention is directed to a method of administering MDCO-2010, or a pharmaceutically acceptable salt or ester thereof, to a subject undergoing surgery, comprising administering a bolus dose of at least about 1 μg/kg of MDCO-2010, or a pharmaceutically acceptable salt or ester thereof, to a subject undergoing surgery, followed by administering a maintenance dose of at least about 1 μg/kg/h of MDCO-2010, or a pharmaceutically acceptable salt or ester thereof, to the subject.

In certain aspects, the method may be used to inhibit, treat or prevent blood loss in a subject to which the compound is administered.

In alternative aspects of this embodiment, the bolus dose is independently at least about 5 μg/kg, at least about 10 μg/kg, about 5 μg/kg, about 11 μg/kg, about 27 μg/kg, between about 5 and 100 μg/kg, or between about 5 and 50 μg/kg of MDCO-2010, or the salt or ester thereof. The maintenance dose is independently at least about 10 μg/kg/h, at least about 30 μg/kg/h, about 12.5 μg/kg/h, about 25 μg/kg/h, about 62.5 μg/kg/h, between about 10 and 200 μg/kg/h, or between about 10 and 100 μg/kg/h of MDCO-2010, or the salt or ester thereof.

In certain aspects of this embodiment, the bolus dose and/or the maintenance dose is administered to the subject via infusion, such as continuous infusion.

In a third embodiment, the present invention is directed to a method of inhibiting, treating or preventing blood loss in a subject undergoing surgery, comprising administering to a subject in need thereof an amount of MDCO-2010, or a pharmaceutically acceptable salt or ester thereof, sufficient to achieve a steady-state plasma concentration of MDCO-2010, or the salt or ester thereof, within about 60 minutes of administration to the subject, thereby inhibiting, treating or preventing blood loss in the subject undergoing surgery.

In certain aspects of this embodiment, the amount of MDCO-2010, or the salt or ester thereof, administered to the subject is at least about 1 μg/kg/h, at least about 10 μg/kg/h, at least about 30 μg/kg/h, about 12.5 μg/kg/h, about 25 μg/kg/h, about 62.5 μg/kg/h, between about 1 and 500 μg/kg/h, between about 10 and 250 μg/kg/h, between about 10 and 200 μg/kg/h or between about 10 and 100 μg/kg/h of MDCO-2010, or the salt or ester thereof.

In certain aspects of this embodiment, MDCO-2010, or the salt or ester thereof, is administered to the subject via infusion, such as continuous infusion.

In an alternative aspect of this embodiment, the amount administered to the subject is a bolus dose of MDCO-2010, or the salt or ester thereof, followed by a maintenance dose of MDCO-2010, or the salt or ester thereof. In this alternative aspect, the bolus dose is independently at least about 1 μg/kg, at least about 5 μg/kg, at least about 10 μg/kg, about 5 μg/kg, about 11 μg/kg, about 27 μg/kg, between about 5 and 100 μg/kg, or between about 5 and 50 μg/kg of MDCO-2010, or the salt or ester thereof. The maintenance dose is independently at least about 1 μg/kg/h, at least about 10 μg/kg/h, at least about 30 μg/kg/h, about 12.5 μg/kg/h, about 25 μg/kg/h, about 62.5 μg/kg/h, between about 10 and 200 μg/kg/h, or between about 10 and 100 μg/kg/h of MDCO-2010, or the salt or ester thereof.

In certain aspects of this embodiment, the steady-state plasma concentration is achieved within about 50 minutes, about 40 minutes, about 30 minutes, about 20 minutes, about 10 minutes, about 5 minutes, or about 1 minute of administration.

In a fourth embodiment, the present invention is directed to a method of inhibiting, treating or preventing blood loss in a subject undergoing surgery, comprising administering to a subject in need thereof an amount of MDCO-2010, or a pharmaceutically acceptable salt or ester thereof, sufficient to achieve a maximum plasma concentration (Cₘₐₓ) of MDCO-2010, or the salt or ester thereof, of not less than about 50 ng/mL in the subject, thereby inhibiting, treating or preventing blood loss in the subject undergoing surgery.

In certain aspects, the maximum plasma concentration (Cₘₐₓ) of MDCO-2010, or the salt or ester thereof, is not less than about 250 ng/mL, not less than about 500 ng/mL, not less than about 750 ng/mL, or not less than about 1000 ng/mL in the subject.

In certain aspects of this embodiment, the amount of MDCO-2010, or the salt or ester thereof, administered to the subject is at least about 1 μg/kg/h, at least about 10 μg/kg/h, at least about 30 μg/kg/h, about 12.5 μg/kg/h, about 25 μg/kg/h, about 62.5 μg/kg/h, between about 1 and 500 μg/kg/h, between about 10 and 250 μg/kg/h, between about 10 and 200 μg/kg/h or between about 10 and 100 μg/kg/h of MDCO-2010, or the salt or ester thereof.

In certain aspects of this embodiment, MDCO-2010, or the salt or ester thereof, is administered to the subject via infusion, such as continuous infusion.

In an alternative aspect of this embodiment, the amount administered to the subject is a bolus dose of MDCO-2010, or the salt or ester thereof, followed by a maintenance dose of MDCO-2010, or the salt or ester thereof. In this alternative aspect, the bolus dose is independently at least about 1 μg/kg, at least about 5 μg/kg, at least about 10 μg/kg, about 5 μg/kg, about 11 μg/kg, about 27 μg/kg, between about 5 and 100 μg/kg, or between about 5 and 50 μg/kg of MDCO-2010, or the salt or ester thereof. The maintenance dose is independently at least about 1 μg/kg/h, at least about 10 μg/kg/h, at least about 30 μg/kg/h, about 12.5 μg/kg/h, about 25 μg/kg/h, about 62.5 μg/kg/h, between about 10 and 200 μg/kg/h, or between about 10 and 100 μg/kg/h of MDCO-2010, or the salt or ester thereof.

Exemplary surgeries in which the methods of the invention may be practiced include cardiothoracic surgeries, including heart, value and aortic surgery, including surgeries requiring cardiopulmonary bypass (CPB), such as coronary artery bypass graft (CABG) surgery. However, it will be recognized that surgery is not limited to cardiothoracic surgeries but can include all types of surgery where blood loss is a concern.

### BRIEF DESCRIPTION OF THE DRAWINGS

FIG. 1 shows the pharmacokinetic profile for MDCO-2010.

FIG. 2 shows inhibition of tPA-induced fibrinolysis by MDCO-2010.

FIG. 3 shows the anticoagulant effect of MDCO-2010 where cohort 1=loading 5 μg/kg, maintenance 12.5 μg/kg/h; cohort 2=loading 11 μg/kg, maintenance 25 μg/kg/h; cohort 3=loading 27 μg/kg, maintenance 62.5 μg/kg/h; cohort 4=loading 47 μg/kg, maintenance 109 μg/kg/h; cohort 5=loading 94 μg/kg, maintenance 218 μg/kg/h.

FIG. 4 shows mean MDCO-2010 concentration vs. time curves (±SD, linear scale) for each cohort, where the cohorts are those of FIG. 3.

FIG. 5 shows mean MDCO-2010 concentration vs. time curves (±SD, semi-logarithmic scale) for each cohort, where the cohorts are those of FIG. 3.

### DETAILED DESCRIPTION OF THE INVENTION

The present invention is generally directed to methods of inhibiting, treating or preventing blood loss in a subject, such as when the subject is undergoing surgery. The methods of the invention are based on the administration of a therapeutically effective amount of MDCO-2010 to a subject. Administration of MDCO-2010 can be used to inhibit, treat and/or prevent blood loss in a subject, such as during surgery.

MDCO-2010 is a synthetic peptidomimetic small molecule. It is an active-site directed reversible inhibitor of serine proteases involved in hemostasis, blocking the proteolytic activity of these enzymes with no requirement for a cofactor. It is a potent inhibitor of its primary targets plasma kallikrein and plasmin. In addition, it inhibits coagulation Factors Xa and XIa as well as the activated Protein C.

MDCO-2010 consists of two unnatural amino acids based on D-phenylpropylglycine and 3-aminomethyl-L-phenylalanine.

Chemical name: 3-({1-[2-(3-Aminomethyl-phenyl)-1-(4-carbamimidoyl-benzylcarbamoyl)-ethylcarbamoyl]-4-phenyl-butylsulfamoyl}-methyl)-benzoic acid

CAS registry number: Not assigned

Molecular formula: C₃₇H₄₂N₆O₆S (free base)

Relative molecular mass: 698.29 g/mol (free base)

### Structural Formula for MDCO-2010

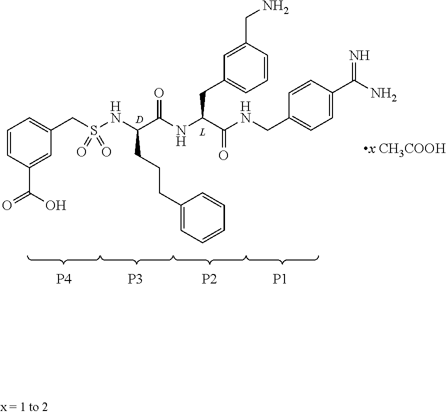

As used herein, all references to MDCO-2010 include MDCO-2010 itself, as well as pharmaceutically acceptable salts and esters of the compound.

MDCO-2010 may be formulated into a clear sterile, isotonic liquid solution for IV infusion, containing MDCO-2010 as anhydrous base. A suitable formulation comprises 1 mg/mL MDCO-2010. In one example, MDCO-2010 is dissolved in 2.5% (w/w) glycerol in water for injection. A pH of between about 4.0 and 7.0 results in the solution. 2.5% glycerol is used due to its isotonic properties and compatibility with the active ingredient. A convenient package provides 50 mL of a 1 mg/mL MDCO-2010 solution in 50 mL Type I glass vials with butyl stoppers and an overseal.

MDCO-2010 solution for infusion in 2.5% glycerol is non-hemolytic, isotonic, and does not change the pH of human plasma upon one-fold or ten-fold dilution with plasma. The solution for infusion is physically (no precipitation of the active ingredient) and chemically stable for at least two weeks at 2 to 8° C., room temperature, and 40° C., either in the dark or in diffuse daylight. The solution for infusion can be diluted two- or ten-fold by volume with either 0.9% NaCl or 5% glucose without occurrence of precipitation of the active ingredient. The dilutions are physically and chemically stable for 18 hours at room temperature.

As indicated above, the present invention is directed to methods of inhibiting, treating or preventing blood loss in a subject undergoing surgery, comprising administering to a subject in need thereof a therapeutically effective amount of MDCO-2010, or a pharmaceutically acceptable salt or ester thereof, thereby inhibiting, treating or preventing blood loss in the subject undergoing surgery.

As used herein, the therapeutically effective amounts of MDCO-2010 that may be used in the methods of the present invention will variety depending on a number of factors, such as the particular procedure being undertaken and the weight of the subject, and will therefore generally be set by an attending physician. However, the following amounts of MDCO-2010 for administration to a subject when practicing methods of the present invention are also acceptable. The therapeutically effective amount of MDCO-2010 thus includes, but is not limited to, administration of at least about: 1, 2, 4, 6, 8, 10, 11, 12, 12.5, 14, 16, 18, 20, 22, 24, 25, 26, 27, 28, 30, 32, 34, 36, 38, 40, 42, 44, 46, 48, 50, 52, 54, 56, 58, 60, 62, 62.5, 64, 66, 68, 70, 72, 74, 76, 78, 80, 82, 84, 86, 88, 90, 92, 94, 96, 98, 100, 110, 120, 130, 140, 150, 160, 170, 180, 190 or 200 μg/kg/h, or more. Alternatively, the therapeutically effective amount of MDCO-2010 includes, but is not limited to, administration of about: 1, 2, 4, 6, 8, 10, 11, 12, 12.5, 14, 16, 18, 20, 22, 24, 25, 26, 27, 28, 30, 32, 34, 36, 38, 40, 42, 44, 46, 48, 50, 52, 54, 56, 58, 60, 62, 62.5, 64, 66, 68, 70, 72, 74, 76, 78, 80, 82, 84, 86, 88, 90, 92, 94, 96, 98, 100, 110, 120, 130, 140, 150, 160, 170, 180, 190 or 200 μg/kg/h, or more. Alternatively, the therapeutically effective amount of MDCO-2010 includes, but is not limited to, administration of between about: 1 and 500, 1 and 400, 1 and 300, 1 and 250, 1 and 200, 1 and 100, 1 and 50, 10 and 500, 10 and 400, 10 and 300, 10 and 250, 10 and 200, 10 and 100, 10 and 75, 10 and 50, 25 and 500, 25 and 400, 25 and 300, 25 and 250, 25 and 200, 25 and 100, 25 and 75, 25 and 50, 40 and 500, 40 and 400, 40 and 300, 40 and 250, 40 and 200, 40 and 100, 40 and 75, or 40 and 50 μg/kg/h.

The therapeutically effective amounts of MDCO-2010 that may be used in the methods of the present invention may also be administered to the subject as a bolus or loading dose, followed by a maintenance dose of MDCO-2010. While the specific dosage amounts will be determined by an attending physician, an acceptable bolus dose includes, but is not limited to, at least about: 1, 2, 4, 6, 8, 10, 11, 12, 12.5, 14, 16, 18, 20, 22, 24, 25, 26, 27, 28, 30, 32, 34, 36, 38, 40, 42, 44, 46, 48, 50, 52, 54, 56, 58, 60, 62, 62.5, 64, 66, 68, 70, 72, 74, 76, 78, 80, 82, 84, 86, 88, 90, 92, 94, 96, 98, 100, 110, 120, 130, 140, 150, 160, 170, 180, 190 or 200 μg/kg of MDCO-2010, or more. An alternative acceptable bolus dose includes, but is not limited to, about: 1, 2, 4, 6, 8, 10, 11, 12, 12.5, 14, 16, 18, 20, 22, 24, 25, 26, 27, 28, 30, 32, 34, 36, 38, 40, 42, 44, 46, 48, 50, 52, 54, 56, 58, 60, 62, 62.5, 64, 66, 68, 70, 72, 74, 76, 78, 80, 82, 84, 86, 88, 90, 92, 94, 96, 98, 100, 110, 120, 130, 140, 150, 160, 170, 180, 190 or 200 μg/kg of MDCO-2010, or more. A further alternative acceptable bolus dose includes, but is not limited to, between about: 1 and 500, 1 and 400, 1 and 300, 1 and 250, 1 and 200, 1 and 100, 1 and 50, 5 and 250, 5 and 200, 5 and 100, 5 and 50, 10 and 500, 10 and 400, 10 and 300, 10 and 250, 10 and 200, 10 and 100, 10 and 75, 10 and 50, 25 and 500, 25 and 400, 25 and 300, 25 and 250, 25 and 200, 25 and 100, 25 and 75, 25 and 50, 40 and 500, 40 and 400, 40 and 300, 40 and 250, 40 and 200, 40 and 100, 40 and 75, or 40 and 50 μg/kg of MDCO-2010.

An acceptable dosage for the maintenance dose following the bolus dose includes, but is not limited to, at least about: 1, 2, 4, 6, 8, 10, 11, 12, 12.5, 14, 16, 18, 20, 22, 24, 25, 26, 27, 28, 30, 32, 34, 36, 38, 40, 42, 44, 46, 48, 50, 52, 54, 56, 58, 60, 62, 62.5, 64, 66, 68, 70, 72, 74, 76, 78, 80, 82, 84, 86, 88, 90, 92, 94, 96, 98, 100, 110, 120, 130, 140, 150, 160, 170, 180, 190 or 200 μg/kg/h of MDCO-2010, or more. Alternatively, an acceptable dosage for the maintenance dose following the bolus dose includes, but is not limited to, about: 1, 2, 4, 6, 8, 10, 11, 12, 12.5, 14, 16, 18, 20, 22, 24, 25, 26, 27, 28, 30, 32, 34, 36, 38, 40, 42, 44, 46, 48, 50, 52, 54, 56, 58, 60, 62, 62.5, 64, 66, 68, 70, 72, 74, 76, 78, 80, 82, 84, 86, 88, 90, 92, 94, 96, 98, 100, 110, 120, 130, 140, 150, 160, 170, 180, 190 or 200 μg/kg/h of MDCO-2010, or more. Alternatively, an acceptable dosage for the maintenance dose following the bolus dose includes, but is not limited to, between about: 1 and 500, 1 and 400, 1 and 300, 1 and 250, 1 and 200, 1 and 100, 1 and 50, 10 and 500, 10 and 400, 10 and 300, 10 and 250, 10 and 200, 10 and 100, 10 and 75, 10 and 50, 25 and 500, 25 and 400, 25 and 300, 25 and 250, 25 and 200, 25 and 100, 25 and 75, 25 and 50, 40 and 500, 40 and 400, 40 and 300, 40 and 250, 40 and 200, 40 and 100, 40 and 75, or 40 and 50 μg/kg/h of MDCO-2010.

The bolus dose and the maintenance dose may each be independently determined. As a result, any bolus dose listed herein may be used in a method that comprises any maintenance dose described herein.

The present invention is also directed to a method of administering MDCO-2010, or a pharmaceutically acceptable salt or ester thereof, to a subject undergoing surgery, comprising administering a bolus dose of at least about 1 μg/kg of MDCO-2010, or a pharmaceutically acceptable salt or ester thereof, to a subject undergoing surgery, followed by administering a maintenance dose of at least about 1 μg/kg/h of MDCO-2010, or a pharmaceutically acceptable salt or ester thereof, to the subject. In certain aspects, the method may be used to inhibit, treat or prevent blood loss in a subject undergoing surgery to which the compound is administered.

In alternatives of this method, an acceptable bolus dose includes, but is not limited to, at least about: 2, 4, 6, 8, 10, 11, 12, 12.5, 14, 16, 18, 20, 22, 24, 25, 26, 27, 28, 30, 32, 34, 36, 38, 40, 42, 44, 46, 48, 50, 52, 54, 56, 58, 60, 62, 62.5, 64, 66, 68, 70, 72, 74, 76, 78, 80, 82, 84, 86, 88, 90, 92, 94, 96, 98, 100, 110, 120, 130, 140, 150, 160, 170, 180, 190 or 200 μg/kg of MDCO-2010, or more. An acceptable bolus dose also includes, but is not limited to, about: 1, 2, 4, 6, 8, 10, 11, 12, 12.5, 14, 16, 18, 20, 22, 24, 25, 26, 27, 28, 30, 32, 34, 36, 38, 40, 42, 44, 46, 48, 50, 52, 54, 56, 58, 60, 62, 62.5, 64, 66, 68, 70, 72, 74, 76, 78, 80, 82, 84, 86, 88, 90, 92, 94, 96, 98, 100, 110, 120, 130, 140, 150, 160, 170, 180, 190 or 200 μg/kg of MDCO-2010, or more. An acceptable bolus dose further includes, but is not limited to, between about: 1 and 500, 1 and 400, 1 and 300, 1 and 250, 1 and 200, 1 and 100, 1 and 50, 5 and 250, 5 and 200, 5 and 100, 5 and 50, 10 and 500, 10 and 400, 10 and 300, 10 and 250, 10 and 200, 10 and 100, 10 and 50, 25 and 500, 25 and 400, 25 and 300, 25 and 250, 25 and 200, 25 and 100, 25 and 50, 40 and 500, 40 and 400, 40 and 300, 40 and 250, 40 and 200, 40 and 100, or 40 and 50 μg/kg of MDCO-2010.

Likewise, in alternatives of this method an acceptable maintenance dose includes, but is not limited to, administration of at least about: 2, 4, 6, 8, 10, 11, 12, 12.5, 14, 16, 18, 20, 22, 24, 25, 26, 27, 28, 30, 32, 34, 36, 38, 40, 42, 44, 46, 48, 50, 52, 54, 56, 58, 60, 62, 62.5, 64, 66, 68, 70, 72, 74, 76, 78, 80, 82, 84, 86, 88, 90, 92, 94, 96, 98, 100, 110, 120, 130, 140, 150, 160, 170, 180, 190 or 200 μg/kg/h of MDCO-2010, or more. An acceptable maintenance dose also includes, but is not limited to, administration of about: 1, 2, 4, 6, 8, 10, 11, 12, 12.5, 14, 16, 18, 20, 22, 24, 25, 26, 27, 28, 30, 32, 34, 36, 38, 40, 42, 44, 46, 48, 50, 52, 54, 56, 58, 60, 62, 62.5, 64, 66, 68, 70, 72, 74, 76, 78, 80, 82, 84, 86, 88, 90, 92, 94, 96, 98, 100, 110, 120, 130, 140, 150, 160, 170, 180, 190 or 200 μg/kg/h of MDCO-2010, or more. An acceptable maintenance dose further includes, but is not limited to, administration of between about: 1 and 500, 1 and 400, 1 and 300, 1 and 250, 1 and 200, 1 and 100, 1 and 50, 10 and 500, 10 and 400, 10 and 300, 10 and 250, 10 and 200, 10 and 100, 10 and 50, 25 and 500, 25 and 400, 25 and 300, 25 and 250, 25 and 200, 25 and 100, 25 and 50, 40 and 500, 40 and 400, 40 and 300, 40 and 250, 40 and 200, 40 and 100, or 40 and 50 μg/kg/h of MDCO-2010.

The bolus dose and the maintenance dose may each be independently determined. As a result, any bolus dose listed herein may be used in a method that comprises any maintenance dose described herein.

The present invention is furthermore directed to a method of inhibiting, treating or preventing blood loss in a subject undergoing surgery, comprising administering to a subject in need thereof an amount of MDCO-2010, or a pharmaceutically acceptable salt or ester thereof, sufficient to achieve a steady-state plasma concentration of MDCO-2010, or the salt or ester thereof, within about 60 minutes of administration to the subject, thereby inhibiting, treating or preventing blood loss in the subject undergoing surgery.

Acceptable amounts of MDCO-2010 associated with this method are those defined above with respect to other methods of the present invention.

This method may also be practiced by achieving the steady-state plasma concentration within about: 58, 56, 54, 52, 50, 48, 46, 45, 44, 42, 40, 38, 36, 35, 34, 32, 30, 28, 26, 24, 22, 20, 18, 16, 15, 14, 12, 10, 8, 6, 5, 4, 3, 2 or 1 minute, or less, of administration.

In addition, the present invention is directed to a method of inhibiting, treating or preventing blood loss in a subject undergoing surgery, comprising administering to a subject in need thereof an amount of MDCO-2010, or a pharmaceutically acceptable salt or ester thereof, sufficient to achieve a maximum plasma concentration (Cₘₐₓ) of MDCO-2010, or the salt or ester thereof, of not less than about 50 ng/mL in the subject, thereby inhibiting, treating or preventing blood loss in the subject undergoing surgery.

This method may also be practiced by administering an amount of MDCO-2010 sufficient to achieve a maximum plasma concentration (Cₘₐₓ) of MDCO-2010 of not less than about: 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975 or 1000 ng/mL, or more, in the subject.

The amount of time needed to achieve the noted Cₘₐₓ need not be limited. However, in acceptable embodiments the amount of time needed to achieve a particular Cₘₐₓ may be about 1, 2, 4, 6, 8, 10, 11, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 120, 130, 140, 150, 160, 170, 180, 190 or 200 minutes, or more.

In each of the methods of the present invention, it is acceptable to administer MDCO-2010 to the subject via infusion, including continuous infusion, such as continuous IV infusion, whether it is a bolus dose, a maintenance dose or simply a therapeutically effective amount of MDCO-2010. However, the skilled artisan will understand that alternative means for administering the compound to a subject may also be acceptable.

Each of the methods of the present invention may also be practiced by administering an ascending or descending dose of MDCO-2010 to the subject. For example, the bolus dose, the maintenance dose or simply a therapeutically effective amount of MDCO-2010 may be administered to a subject via infusion whereby the concentration of the compound in the infusion increases or decreases over the duration of the infusion or whereby the flow rate of the infusion increases or decreases over the duration of the infusion. The concentration of the compound may increase or decrease by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% over the duration of the infusion. The flow rate of the infusion may increase or decrease by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% over the duration of the infusion.

The period of time over which the bolus dose is administered when the various methods recited herein are practiced will vary and it will be set by an attending physician. However, when administered via infusion acceptable periods of time include, but are not limited to, a period of about 1, 2, 3, 4, 5, 6, 7, 8, 9, 10, 11, 12, 13, 14, 15, 16, 17, 18, 19 or 20 minutes, or more.

When administered by infusion, the flow rate for the maintenance dose or other therapeutically effective amounts of MDCO-2010 of the present invention will again vary and be set by an attending physician. However, acceptable flow rates include, but are not limited to, about: 1, 2, 4, 6, 8, 10, 11, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 or 80 mL/h, or more.

The time point at which administration of MDCO-2010 to a subject undergoing surgery will begin (whether a bolus dose, a maintenance dose or simply a therapeutically effective amount of MDCO-2010) depends on factors including the characteristics of the surgical procedure to be performed. Under most circumstances, administration will begin at least about 2, 4, 6, 8, 10, 12, 14, 16, 18 or 20 minutes, or more, prior to the start of the surgical procedure. As used herein, “the start of the surgical procedure” is simply that point in time where the subject is placed at an increased risk of bleeding, such as, but not limited to, when the skin is incised or when a cardiopulmonary bypass procedure begins. The time point at which administration of MDCO-2010 will end again depends on factors including the characteristics of the surgical procedure to be performed. Under most circumstances, administration will end less than about 2, 4, 6, 8, 10, 12, 14, 16, 18, 20, 22, 24, 26, 28 or 30 minutes, or more, after the surgical procedure has been completed.

When the methods of the present invention include administration via both bolus and maintenance dosing, the period of time between the completion of bolus administration and the start of maintenance is preferably less than about 10, 9, 8, 7, 6, 5, 4, 3, 2 or 1 minute, or less than about 60, 50, 40, 30, 20 or 10 seconds. In one embodiment, there is no interruption between bolus and maintenance dosing, with administration of the maintenance dose beginning as administration of the bolus dose ends.

The subjects upon which the methods of the present invention may be practice include, but are not limited to, humans, primates, and other mammals, such as dogs, cats, pigs, horses, sheep cattle, and goats.

In each of the methods of the present invention, the subject is undergoing surgery. Exemplary surgeries include all forms of cardiothoracic surgery, including, but not limited to, heart, value and aortic surgery. Each of the methods of the invention is particularly well-suited for use in conjunction with surgery requiring cardiopulmonary bypass (CPB). CPB is used to maintain blood flow and to facilitate tissue oxygenation during surgical procedures with cardioplegia, such as coronary artery bypass grafting (CABG), cardiac valve replacement or repair, thoracic aortic aneurysm repair, and heart or heart/lung transplantation. However, it will be recognized that surgery is not limited to cardiothoracic surgeries but can include other types of surgery where blood loss is a concern.

### Examples

A Phase IIa, single-center, double-blind, placebo-controlled dose-escalation study was performed to investigate MDCO-2010 in patients undergoing elective, primary CABG (coronary artery bypass graft) surgery.

After independent ethics committee approval and written informed consent, thirty two patients underwent CABG surgery (patient characteristics shown in Table 1) through midline sternotomy using minimized extracorporeal circulation (MECC, priming volume of 600 mL).

TABLE 1

| | Total cohort (n = 32) |
|---|---|
| Age [years] | 63.9 ± 9.2 |
| Gender (no. of male/female patients) | 25/5 |
| Body mass index [kg/m²] | 26.3 ± 3.5 |
| LV Ejection fraction [%] | 62.8 ± 8.0 |
| EuroSCORE (additive) | 2.5 ± 1.5 |
| No. of grafts | 3.3 ± 0.8 |
| Aortic cross clamp time [min] | 40.8 ± 12.4 |
| CPB time [min] | 63.2 ± 16.5 |
| Infusion duration [min] | 117.6 ± 26.0 |
| Surgery to hospital discharge [days] | 7.4 ± 1.6 |

Continuous variables reported as mean ± SD

Initial heparin dosing (400 units/kg) and heparin maintenance was guided by the Hepcon HMS, with a kaolin ACT target of 480 seconds. Immediately after heparin bolus, study drug administration was initiated with a loading (bolus) dose followed by a continuous infusion of the maintenance dose via a central venous catheter until sternal closure. Patients were randomized in a 3:1 ratio to receive either one of five MDCO-2010 doses (continuous infusion of 12.5, 25, 62.5, 109 or 219 μg/kg/h maintenance dose; 24 patients) or saline as placebo (8 patients).

### Cohort 1:

loading dose 5 μg/kg, maintenance dose 12.5 μg/kg/h; pump prime 0.02 mg; 3 patients

### Cohort 2:

loading dose 11 μg/kg, maintenance dose 25 μg/kg/h; pump prime 0.04 mg; 3 patients

### Cohort 3:

loading dose 27 μg/kg, maintenance dose 62.5 μg/kg/h; pump prime 0.09 mg; 6 patients

### Cohort 4:

loading dose 47 μg/kg, maintenance dose 109 μg/kg/h; pump prime 0.18 mg; 6 patients

### Cohort 5:

loading dose 94 μg/kg, maintenance dose 219 μg/kg/h; pump prime 0.35 mg; 6 patients The loading dose was administered over a five minute period of time in a 10 mL infusion. The maintenance dose was administered as a continuous infusion with a constant flow rate of 23 mL/h for the duration of procedure. MDCO-2010 was also added to the pump prime fluid to the targeted plasma concentration. The pump prime was supplied as a 10 mL load.

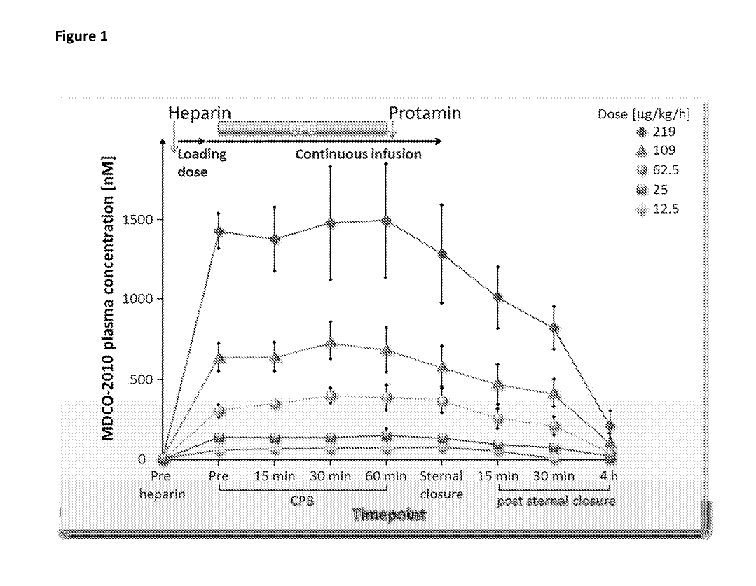

Pharmacokinetic (PK) analysis showed a linear dose-proportional increase of mean MDCO-2010 plasma concentrations. Plasma levels were stable during the infusion (FIG. 1), eliminated with a terminal half-life of 1 hour and abated by 85% within 4 hours of the end of infusion.

No differences in troponin T, CK-MB, prothrombin fragment F1+2, interleukins IL-6 and IL-10, or ALAT, ASAT, serum creatinine were observed between patients treated with MDCO-2010 vs. placebo (results not shown).

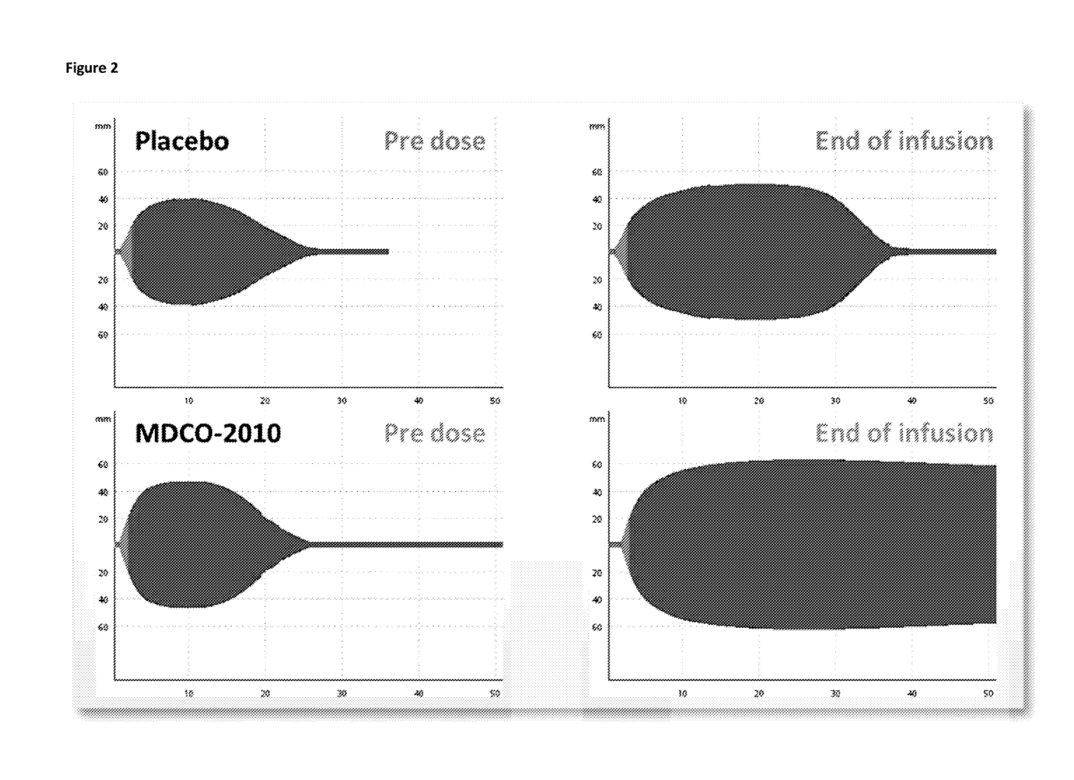

MDCO-2010 exhibited a dose-dependent antifibrinolytic effect as demonstrated by suppression of D-dimer generation and inhibition of tPA-induced lysis (FIG. 2).

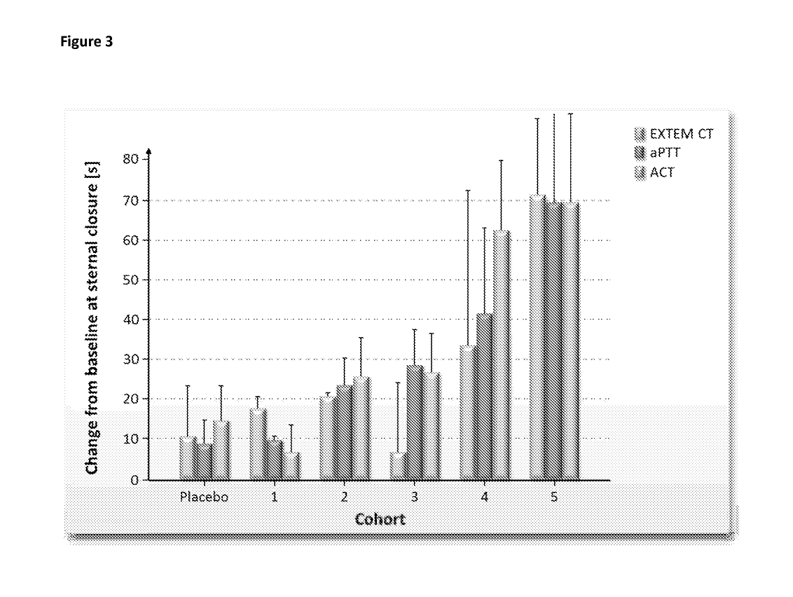

MDCO-2010 also showed a dose-related prolongation of ACT, aPTT and rotation thrombelastometry (ROTEM) coagulation times (FIG. 3), indicating an anticoagulant effect involving both intrinsic and extrinsic pathways.

Seven out of 32 patients (22%) received allogeneic blood products perioperatively. No patients required surgical re-exploration for bleeding or pericardial tamponade. All patients were discharged from ICU on the first postoperative day and had a mean postoperative hospitalization time of 7.4 days. No deaths occurred within the first 30 postoperative days.

Chest tube drainage, incidence and volume of RBC transfusions (according to institutional transfusion guideline) were significantly lower with MDCO-2010 vs. placebo (Table 2).

TABLE 2

| | Placebo | Cohort 1 | Cohort 2 | Cohort 3 | Cohort 4 | Cohort 5 |
|---|---|---|---|---|---|---|
| | (N = 8) | (N = 3) | (N = 3) | (N = 6) | (N = 6) | (N = 6) |
| 12 hour postoperative | 900 ± 349 | 450 ± 172 | 595 ± 125 | 350* ± 39 | 350* ± 106 | 360* ± 220 |
| chest tube drainage, | | | | | | |
| median ± SD [mL] | | | | | | |
| Patients receiving | 4/8 | 0/3 | 0/3 | 1/6 | 1/6 | 2/6† |
| transfusions | | | | | | |
| Average number of | 2.00 | 0 | 0 | 0.33 | 0.17 | 0.67† |
| transfusions per patient | | | | | | |

*p < 0.025 vs. placebo
†1 patient in cohort 5 received additional heparin without protamine reversal

### PK Profile

Nominal blood sampling times for PK analysis were pre-dose (T1, pre-heparin) and at 10 min (pre-CBP), 15 min, 30 min, 60 min (or just before cessation of CPB), at the end of infusion (sternal closure), 15 min, 30 min, and at 4 hours post-end of infusion. Actual times were calculated for each patient and used for PK analysis.

MDCO-2010 was measured in plasma by liquid chromatography with tandem mass spectrometry (LC/MS/MS).

Plasma concentrations versus time data were analyzed by non-compartmental analysis using the program WinNonlin Professional Version 5.3 (Pharsight, Mountain View, Calif., USA). Actual sampling times were used for the evaluation.

The following parameters were determined:
AUC₀₋ₜₗₐₛₜ=area under the MDCO-2010 concentration versus time curve from dosing time to the last time point with a value above the lower limit of quantitation (LLOQ), calculated by linear/log trapezoidal method which uses the linear trapezoidal rule up to Cₘₐₓ and log trapezoidal rule for the remainder of the curve
AUC_{0-inf}=area under the concentration-time curve from time 0 to the last measured time point with a concentration above the LLOQ; calculated as AUC₀₋ₜₗₐₛₜ+AUCₑₓₚₒₗ, where AUCₑₓₚₒₗ=Cₗₐₛₜ/λ
Css=steady-state plasma concentration calculated by taking the average of the concentrations in T4 to T7
Cₘₐₓ=maximum concentration, directly taken from measured values
t_{1/2}=apparent terminal elimination half-life determined by log-linear regression
CL=total body clearance
Vₛₛ=volume of distribution at steady state

Descriptive statistics (number of subjects, arithmetic means, standard deviation (SD), geometric means, medians, minimum and maximum values) were determined for all PK variables, separately for each treatment. Values below the LLOQ of the assay were taken as zero for descriptive statistics of concentrations. For AUC calculations, values below LLOQ were taken as zero if no quantifiable concentrations were found before the value, as missing if quantifiable concentrations were found before and after the value, and also as missing if quantifiable concentrations were found before, but not after the value. Mean (±SD) concentration time plots were provided for each treatment group using linear and semi-logarithmic scale.

Dose proportionality was assessed by the power model using SAS Version 9 (SAS Institute Inc., Cary, N.C.).

The mean values for PK parameters per dose group (mean (±SD) pharmacokinetic variables of MDCO-2010 administered via IV infusion during CABG surgery based on non-compartmental analysis) are summarized in Table 3.

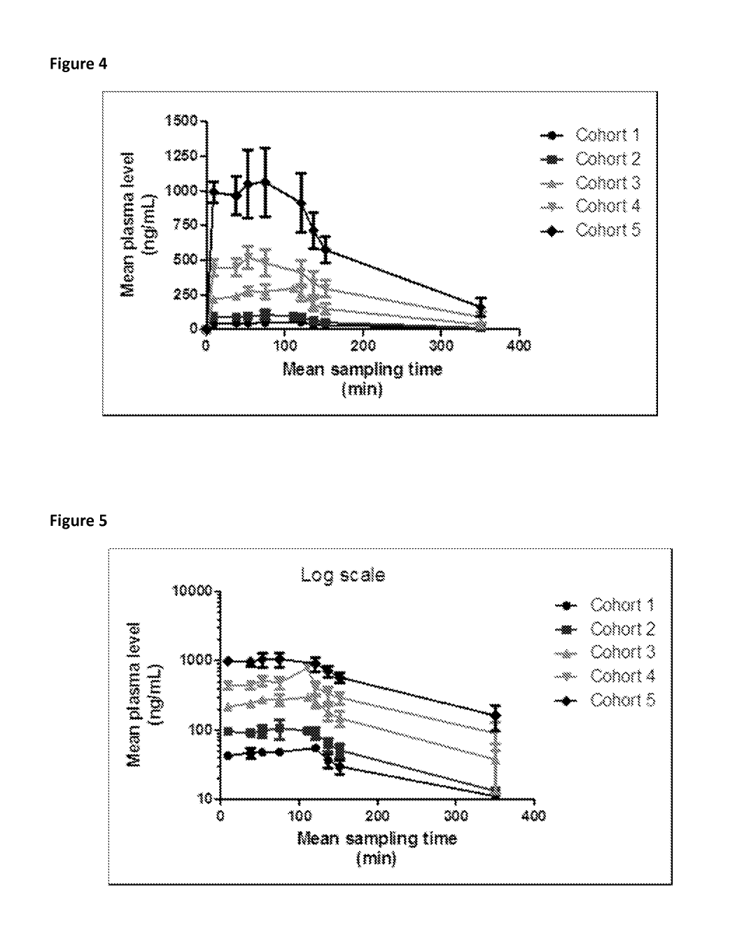

MDCO-2010 plasma levels increased rapidly with the start of bolus infusion and maintained stable levels during the infusion (FIGS. 4 and 5: Linear and log scale mean MDCO-2010 concentration vs. time curves for each cohort). Following an approximately 100 minute infusion period, MDCO-2010 levels are rapidly decreased with a mean value for the short elimination half-life of about 83 minutes (range of 61 to 102 minutes). Total body clearance slightly decreased with dose from 220±78 mL/min to 153±19 mL/min (1.88 mg and 38.01 mg total dose, respectively). Volume of distribution (Vₛₛ) values were higher than that of the putative central compartment (vascular system) at all dose levels (range of 12 to 17 liters), which indicates additional distribution to other physiologic compartments or tissues. The Vₛₛ corresponds approximately to the extracellular fluid volume.

TABLE 3

| | Mean | | | | | | | |
|---|---|---|---|---|---|---|---|---|
| | Dose | Cₘₐₓ | Cₛₛ | AUCₗₐₛₜ | AUC_{inf} | t_{1/2} | Cl | Vss |
| Cohort | (mg) | (ng/mL) | (ng/mL) | (min*ng/mL) | (min*ng/mL) | (min) | (mL/min) | (mL) |
| 1 | 1.88 | 54.80 | 50.06 | 7508.12 | 9132.17 | 61.24 | 219.92 | 12696.35 |
| | (0.25) | (4.91) | (3.82) | (2554.45) | (2840.98) | (49.93) | (77.64) | (6022.36) |
| 2 | 4.99 | 114.27 | 96.31 | 16675.78 | 20034.50 | 72.70 | 262.95 | 16118.69 |
| | (1.17) | (19.51) | (20.37) | (8089.54) | (7553.80) | (22.95) | (63.93) | (2795.26) |
| 3 | 12.33 | 287.67 | 268.21 | 54284.06 | 59471.05 | 75.52 | 199.42 | 12304.47 |
| | (1.95) | (40.05) | (40.37) | (16244.31) | (18812.88) | (24.77) | (93.98) | (5436.22) |
| 4 | 19.57 | 563.50 | 478.86 | 98247.70 | 112877.91 | 104.16 | 179.22 | 17382.19 |
| | (5.22) | (128.84) | (101.51) | (28840.21) | (36453.61) | (26.08) | (39.91) | (2749.61) |
| 5 | 38.01 | 1147.00 | 1009.17 | 217352.42 | 243469.88 | 102.93 | 153.04 | 15055.91 |
| | (14.20) | (165.84) | (203.55) | (52036.55) | (66660.89) | (29.71) | (19.48) | (6006.20) |

This first-in-patient study demonstrated predictable pharmacokinetics and an acceptable safety profile of escalating MDCO-2010 doses in primary CABG surgery. Anticipated pharmacodynamic effects on antifibrinolytic and anticoagulant markers were observed. MDCO-2010 showed linear, predictable plasma pharmacokinetics with rapid clearance. MDCO-2010 was associated with significantly reduced 12-hour chest tube drainage and less transfusion requirement.

All documents, books, manuals, papers, patents, published patent applications, guides, abstracts and other reference materials cited herein, including GenBank Accession Numbers, are incorporated by reference in their entirety. While the foregoing specification teaches the principles of the present invention, with examples provided for the purpose of illustration, it will be appreciated by one skilled in the art from reading this disclosure that various changes in form and detail can be made without departing from the true scope of the invention.

1. A method of inhibiting, treating or preventing blood loss in a subject undergoing surgery, comprising administering to a subject in need thereof a therapeutically effective amount of MDCO-2010, or a pharmaceutically acceptable salt or ester thereof, thereby inhibiting, treating or preventing blood loss in the subject undergoing surgery.

2. (canceled)

3. The method of claim 1, wherein the therapeutically effective amount is administration via infusion of between about 10 and 200 μg/kg/h of MDCO-2010, or a pharmaceutically acceptable salt or ester thereof.

4. (canceled)

5. The method of claim 1, wherein the subject is undergoing surgery requiring cardiopulmonary bypass (CPB) or undergoing coronary artery bypass graft (CABG) surgery.

6. (canceled)

7. The method of claim 1, wherein the therapeutically effective amount is administration via infusion of a bolus dose of between about 5 and 100 μg/kg of MDCO-2010, or a pharmaceutically acceptable salt or ester thereof, followed by a maintenance dose of between about 10 and 200 μg/kg/h of MDCO-2010, or a pharmaceutically acceptable salt or ester thereof.

8. (canceled)

9. The method of claim 7, wherein the bolus dose is about 5 μg/kg or about 11 μg/kg.

10. (canceled)

11. The method of claim 7, wherein the maintenance dose is about 12.5 μg/kg/h or about 25 μg/kg/h.

12. (canceled)

13. A method of inhibiting, treating or preventing blood loss in a subject undergoing surgery, comprising administering to a subject in need thereof an amount of MDCO-2010, or a pharmaceutically acceptable salt or ester thereof, sufficient to achieve a steady-state plasma concentration of MDCO-2010, or the salt or ester thereof, within about 60 minutes of administration, thereby inhibiting, treating or preventing blood loss in the subject undergoing surgery.

14. (canceled)

15. The method of claim 13, wherein the amount is administration via infusion of between about 10 and 200 μg/kg/h of MDCO-2010, or a pharmaceutically acceptable salt or ester thereof.

16. (canceled)

17. The method of claim 13, wherein the subject is undergoing surgery requiring cardiopulmonary bypass (CPB) or undergoing coronary artery bypass graft (CABG) surgery.

18. (canceled)

19. The method of claim 13, wherein the steady-state plasma concentration is achieved within about 50 minutes, about 40 minutes, about 30 minutes, about 20 minutes, about 10 minutes or about 5 minutes of administration.

20. The method of claim 13, wherein the steady-state plasma concentration is achieved within about 1 minute of administration.

21. The method of claim 13, wherein the amount is administration via infusion of a bolus dose of between about 5 and 100 μg/kg of MDCO-2010, or a pharmaceutically acceptable salt or ester thereof, followed by a maintenance dose of between about 10 and 200 μg/kg/h of MDCO-2010, or a pharmaceutically acceptable salt or ester thereof.

22. The method of claim 13, wherein the amount is administration via infusion of a bolus dose of between about 5 and 50 μg/kg of MDCO-2010, or a pharmaceutically acceptable salt or ester thereof, followed by a maintenance dose of between about 10 and 100 μg/kg/h of MDCO-2010, or a pharmaceutically acceptable salt or ester thereof.

23. A method of inhibiting, treating or preventing blood loss in a subject undergoing surgery, comprising administering to a subject in need thereof an amount of MDCO-2010, or a pharmaceutically acceptable salt or ester thereof, sufficient to achieve a maximum plasma concentration (Cₘₐₓ) of MDCO-2010, or the salt or ester thereof, of not less than about 50 ng/mL in the subject, thereby inhibiting, treating or preventing blood loss in the subject undergoing surgery.

24. (canceled)

25. The method of claim 23, wherein the amount is administration via infusion of between about 10 and 200 μg/kg/h of MDCO-2010, or a pharmaceutically acceptable salt or ester thereof.

26. (canceled)

27. The method of claim 23, wherein the subject is undergoing surgery requiring cardiopulmonary bypass (CPB) or undergoing coronary artery bypass graft (CABG) surgery.

28. (canceled)

29. The method of claim 23, wherein the Cₘₐₓ is not less than about 100 ng/mL, about 250 ng/mL, about 500 ng/mL, about 750 ng/mL or about 1000 ng/mL.

30. The method of claim 23, wherein the Cₘₐₓ is achieved within about 50 minutes, about 40 minutes, about 30 minutes, about 20 minutes, about 10 minutes, about 5 minutes or about 1 minute of administration.

31. The method of claim 23, wherein the amount is administration via infusion of a bolus dose of between about 5 and 100 μg/kg of MDCO-2010, or a pharmaceutically acceptable salt or ester thereof, followed by a maintenance dose of between about 10 and 200 μg/kg/h of MDCO-2010, or a pharmaceutically acceptable salt or ester thereof.

32. (canceled)
